(19) United States
(12) Patent Application Publication
(10) Pub. No.: US 2022/0287948 A1
(43) Pub. Date: Sep. 15, 2022

(54) FILM-FORMING COMPOSITION

(71) Applicant: KAO CORPORATION, Tokyo (JP)

(72) Inventor: Nobuyuki ASAMI, Odawara-shi (JP)

(73) Assignee: KAO CORPORATION, Tokyo (JP)

(21) Appl. No.: 17/633,129

(22) PCT Filed: Aug. 6, 2020
(86) PCT No.: PCT/JP2020/030201

§ 371 Date: Feb. 4, 2022

(30) Foreign Application Priority Data
Aug. 7, 2019 (JP) 2019-145786

(51) Int. Cl. A61K 8/81; A61K 8/34; A61Q 1/00; A61Q 19/00; A61K 8/02

(52) U.S. Cl. CPC A61K 8/8135; A61K 8/34; A61Q 1/00; A61Q 19/007; A61K 8/027; A61K 2800/83

(57) ABSTRACT

Provided is a film-forming composition directly on the skin by electrostatic spraying, the composition being capable of forming a coating film having excellent skin compatibility, adhesion, followability to motion of the skin, and durability against moisture. A film-forming composition, for forming a coating film composed of fibers directly on the skin by electrostatic spraying, wherein the composition comprises the following components (a), (b) and (c), and the coating film formed from the film-forming composition has an elastic modulus of 1×10⁴ N/m² or more and 9×10⁶ N/m² or less:
(a) a polymer having film-forming ability;
(b) one or more volatile substances selected from the group consisting of an alcohol and a ketone; and
(c) 2 mass % or more and 30 mass % or less of a non-volatile oil having a log S of −7 or more and less than 0.

### FIELD OF THE INVENTION

The present invention relates to a composition for forming a coating film directly on the skin by electrostatic spraying.

### BACKGROUND OF THE INVENTION

Various methods are known for forming a coating film by electrostatic spraying. For example, Patent Literature 1 discloses a method for treating skin including electrostatically spraying a composition on the skin. The composition used in this method contains a liquid insulating substance, an electrically conducting substance, a particulate powder substance, and a thickener. This composition is typically used as cosmetic products containing a pigment and skincare compositions. Specifically, the composition is used as a makeup foundation. That is, Patent Literature 1 mainly aims to wear a makeup on the skin by electrostatically spraying a makeup foundation for a cosmetic purpose.

Patent Literature 2 discloses a disposable cartridge to be used for an electrostatic spraying apparatus of a cosmetic product. This electrostatic spraying apparatus is a hand-held self-contained style. This electrostatic spraying apparatus is used to spray a makeup foundation as in Patent Literature 1 described above.

Patent Literature 3 discloses a method for enhancing coating film adhesion by applying a solution before or after forming a coating film on the surface of the skin by an electrostatic spraying method.

(Patent Literature 1) JP-A-2006-104211

(Patent Literature 2) JP-A-2003-507165

(Patent Literature 3) JP-A-2017-78062

### SUMMARY OF THE INVENTION

The present invention relates to a film-forming composition, for forming a coating film composed of fibers directly on the skin by electrostatic spraying, wherein the composition comprises the following components (a), (b) and (c), and the coating film formed from the film-forming composition has an elastic modulus of 1×10⁴ N/m² or more and 9×10⁶ N/m² or less:

(a) a polymer having film-forming ability;

(b) one or more volatile substances selected from the group consisting of an alcohol and a ketone; and

(c) 2 mass % or more and 30 mass % or less of a non-volatile oil having a log S of −7 or more and less than 0.

One embodiment of the present invention relates to a film-forming composition, for forming a coating film composed of fibers directly on the skin by electrostatic spraying, wherein the composition comprises the following components (a), (b) and (c), and the coating film formed from the film-forming composition has an elastic modulus of 1×10⁴ N/m² or more and 9×10⁶ N/m² or less:

(a) 4 mass % or more and 35 mass % or less of a polymer having film-forming ability;

(b) 45 mass % or more and 95 mass % or less of one or more volatile substances selected from the group consisting of an alcohol and a ketone; and

(c) 2 mass % or more and 30 mass % or less of a non-volatile oil having a log S of −7 or more and less than 0.

One embodiment of the present invention relates to a method for producing a coating film, for forming a coating film on a surface of a coating film formation subject, comprising an electrostatically spraying step for forming a coating film composed of deposits comprising fibers by directly electrostatically spraying a composition on the coating film formation subject, the composition comprising the following components (a), (b) and (c), the coating film formed from the composition having an elastic modulus of 1×10⁴ N/m² or more and 9×10⁶ N/m² or less:

(a) 4 mass % or more and 35 mass % or less of a polymer having film-forming ability;

(b) 45 mass % or more and 95 mass % or less of one or more volatile substances selected from the group consisting of an alcohol and a ketone; and

(c) 2 mass % or more and 30 mass % or less of a non-volatile oil having a log S of −7 or more and less than 0.

One embodiment of the present invention relates to a cosmetic comprising a film-forming composition, for forming a coating film composed of fibers directly on the skin by electrostatic spraying, wherein the composition comprises the following components (a), (b) and (c), and the coating film formed from the film-forming composition has an elastic modulus of 1×10⁴ N/m² or more and 9×10⁶ N/m² or less:

(a) 4 mass % or more and 35 mass % or less of a polymer having film-forming ability;

(b) 45 mass % or more and 95 mass % or less of one or more volatile substances selected from the group consisting of an alcohol and a ketone; and

(c) 2 mass % or more and 30 mass % or less of a non-volatile oil having a log S of −7 or more and less than 0.

### BRIEF DESCRIPTION OF DRAWINGS

FIG. 1 illustrates a diagram showing a structure of the electrostatic spraying apparatus preferably used in the present invention.

FIG. 2 illustrates a schematic view showing a fashion for implementing electrostatic spraying using the electrostatic spraying apparatus.

### DETAILED DESCRIPTION OF THE INVENTION

When a coating film is formed on the skin by implementing electrostatic spraying in accordance with the methods described in Patent Literatures 1 and 2, the adhesion between the skin and the coating film formed by electrostatic spraying is not sufficient and hence the coating film may be damaged or peeled off due to motion of the skin and moisture. Alternatively, when a coating film is formed on the skin by the method described in Patent Literature 3, the adhesion of the coating film is enhanced to a certain extent but followability to further motion of the skin and durability against moisture are demanded, and skin compatibility and a favorable feel are also demanded when the electrostatic spraying is conducted on the skin to which a cosmetic has been applied in advance.

The present invention relates to a composition for forming a coating film directly on the skin by electrostatic spraying, wherein the composition is capable of forming a coating film having excellent skin compatibility, adhesion, followability to motion of the skin, and durability against moisture.

Accordingly, the present inventor conducted various studies on electrostatic spraying compositions used for forming a coating film on the skin instead of a composition applied before or after forming a coating film on the skin by electrostatic spraying, and found that when a non-volatile oil having a specific log S value is used in addition to a polymer having film-forming ability and a volatile substance, the coating film formed directly on the skin by electrostatic spraying has an excellent elastic modus and becomes excellent in skin compatibility, adhesion, followability to motion of the skin, and durability against moisture. Thereby the present inventor completed the present invention.

When a coating film composed of fibers is formed directly on the skin by electrostatic spraying using the composition of the present invention, the obtained coating film has a favorable elastic modulus, and is excellent in skin compatibility, adhesion, followability to motion of the skin, and durability against moisture.

Additionally, when electrostatic spraying is implemented on the skin to which a cosmetic has been applied in advance or when a cosmetic is applied after a coating film is formed on the skin by electrostatic spraying, compatibility between the coating film and the skin is extremely favorable. The elastic modulus of the coating film obtained using the composition of the present invention is preferably 1×10⁷ N/m² or less, and particularly preferably 1×10⁴ N/m² or more and 9×10⁶ N/m² or less from the viewpoints of the adhesion of the coating film and the followability to motion of the skin.

The film-forming composition according to the present invention is a composition for forming a coating film composed of fibers directly on the skin by electrostatic spraying and contains the components (a), (b) and (c) described above. The coating film composed of fibers in the present invention means a coating film containing the fibers of the component (a) and may also be a coating film in which, for example, a liquid substance is present around the fibers other than the fibers. The electrostatic spraying is preferably electrospinning.

The polymer having film-forming ability serving as the component (a) is a substance generally soluble in the (b) volatile substance. Soluble herein refers to a state in which, when the component (a) and the component (b) are mixed, the component (a) is in a dispersed state in the component (b) at 20° C. and such a dispersed state is a visually homogenous state, and preferably a visually transparent or semitransparent state. The film-forming ability of the present invention is preferably fiber-forming ability.

For the polymer having film-forming ability, a suitable polymer may be used in accordance with the property of the (b) volatile substance. Specifically, the polymers having film-forming ability are roughly categorized into water-soluble polymers and water-insoluble polymers. The “water-soluble polymer” in the present specification refers to a polymer having a property that, under environment of 1 atm and 23° C., when 1 g of the polymer is immersed in 10 g of ion exchange water for 24 hours, 0.5 g or more of the immersed polymer is dissolved in water. Meanwhile, the “water-insoluble polymer” in the present specification refers to a polymer having a property that, under an environment of 1 atm and 23° C., when 1 g of the polymer is immersed in 10 g of ion exchange water for 24 hours, only less than 0.5 g of the immersed polymer is dissolved in water.

Examples of the water-soluble polymer having film-forming ability include mucopolysaccharides such as pullulan, hyaluronic acid, chondroitin sulfate, poly-γ-glutamic acid, modified cornstarch, β-glucan, glucooligosaccharide, heparin, and keratosulfate, natural macromolecules such as cellulose, pectin, xylan, lignin, glucomannan, galacturonic acid, psyllium seed gum, tamarind seed gum, gum arabic, gum tragacanth, water-soluble soybean polysaccharide, alginic acid, carrageenan, laminaran, agarose, fucoidan, methyl cellulose, hydroxypropyl cellulose, and hydroxypropyl methylcellulose, synthetic macromolecules such as a partially saponified polyvinyl alcohol (when not used in combination with a crosslinking agent), a low saponified polyvinyl alcohol, polyvinylpyrrolidone (PVP), polyethylene oxide, and sodium polyacrylate. These water-soluble polymers can be used singly or two or more can be used in combination. Of these water-soluble polymers, it is preferable to use pullulan, and synthetic macromolecules such as a partially saponified polyvinyl alcohol, a low saponified polyvinyl alcohol, polyvinylpyrrolidone, and polyethylene oxide from a viewpoint of easy production of a coating film. When polyethylene oxide is used as the water-soluble polymer, a number average molecular weight thereof is preferably 50 000 or more and 3 000 000 or less, and more preferably 100 000 or more and 2 500 000 or less.

Examples of the water-insoluble polymer having film-forming ability include a completely saponified polyvinyl alcohol insolubilizable after a coating film is formed, a partially saponified polyvinyl alcohol crosslinkable after a coating film is formed when used in combination with a crosslinking agent, oxazoline-modified silicones such as a poly(N-propanoylethyleneimine)graft-dimethylsiloxane/γ-aminopropylmethylsiloxane copolymer, a polyvinyl acetal diethylamino acetate, Zein (main component of corn protein), a polyester, a polylactic acid (PLA), an acrylic resin such as a polyacrylonitrile resin, and a polymethacrylic acid resin, and a polystyrene resin, a polyvinyl butyral resin, an alkyl acetalized polyvinyl alcohol, a polyethylene terephthalate resin, a polybutylene terephthalate resin, a polyurethane resin, a polyamide resin, a polyimide resin, and a polyamideimide resin.

These water-insoluble polymers can be used singly or two or more can be used in combination. Of these water-insoluble polymers, it is preferable to use a completely saponified polyvinyl alcohol insolubilizable after a coating film is formed, a partially saponified polyvinyl alcohol crosslinkable after a coating film is formed when used in combination with a crosslinking agent, a polyvinyl butyral resin, an alkyl acetalized polyvinyl alcohol, a polyurethane resin, and oxazoline-modified silicones such as a poly(N-propanoylethyleneimine)graft-dimethylsiloxane/γ-aminopropylmethylsiloxane copolymer, a polyvinyl acetal diethylamino acetate, and Zein.

The component (a) is preferably a water-insoluble polymer having film-forming ability, and more preferably one or more selected from the group consisting of a partially saponified polyvinyl alcohol, a low saponified polyvinyl alcohol, a completely saponified polyvinyl alcohol, a polyvinyl butyral resin, an alkyl acetalized polyvinyl alcohol, a polyurethane resin, a polymethacrylic acid resin, an oxazoline-modified silicone, a polyvinyl acetal diethylamino acetate, and a polylactic acid.

A content of the component (a) in the film-forming composition according to the present invention is preferably 4 mass % or more, more preferably 5 mass % or more, further more preferably 6 mass % or more, and even more preferably 8 mass % or more. Additionally, it is preferably 35 mass % or less, more preferably 30 mass % or less, further more preferably 25 mass % or less, and even more preferably 20 mass % or less. The content of the component (a) in the film-forming composition is preferably 4 mass % or more and 35 mass % or less, more preferably 4 mass % or more and 30 mass % or less, further more preferably 6 mass % or more and 25 mass % or less, and even more preferably 8 mass % or more and 20 mass % or less. When the component (a) is contained in this proportion in the film-forming composition, an intended coating film can be efficiently formed and a coating film composed of fibers can be formed stably.

The volatile substance serving as the component (b) is a substance having volatility in a liquid state. The component (b) in the film-forming composition is formulated for the purpose of finally forming a dry coating film, wherein the film-forming composition placed in the electric field is sufficiently charged, subsequently discharged toward the skin from a nozzle tip, a charge density of the film-forming composition becomes excess as the component (b) evaporates, and component (b) further evaporates while broken down by Coulomb repulsion. For this purpose, the volatile substance has a vapor pressure at 20° C. of preferably 0.01 kPa or more and 106.66 kPa or less, more preferably 0.13 kPa or more and 66.66 kPa or less, further more preferably 0.67 kPa or more and 40.00 kPa or less, further more preferably 1.33 kPa or more and 40.00 kPa or less, and even more preferably 2.40 kPa or more and 40.00 kPa or less.

Among the (b) volatile substances, for example, monovalent chain fatty alcohols, monovalent cyclic fatty alcohols, and monovalent aromatic alcohols are preferably used as the alcohol. Examples of the monovalent chain fatty alcohol include straight chain or branched chain alcohols having 1 to 6 carbon atoms, examples of the monovalent cyclic fatty alcohol include cyclic fatty alcohols having 4 to 6 carbon atoms, and examples of the monovalent aromatic alcohol include benzyl alcohol and phenyl ethyl alcohol. Specific examples thereof include methanol, ethanol, isopropyl alcohol, n-propyl alcohol, n-butyl alcohol, 2-butyl alcohol, isobutyl alcohol, 2-methyl-2-propyl alcohol, n-pentanol, 2-pentanol, 3-pentanol, 2-methyl-1-butyl alcohol, 2-methyl-2-butyl alcohol, 3-methyl-1-butyl alcohol, 3-methyl-2-butyl alcohol, neopentyl alcohol, n-hexanol, 2-hexanol, 3-hexanol, 2-methyl-1-pentanol, 3-methyl-1-pentanol, 4-methyl-1-pentanol, 2-methyl-2-pentanol, 3-methyl-2-pentanol, 4-methyl-2-pentanol, 2-methyl-3-pentanol, 3-methyl-3-pentanol, 2,2-dimethyl-1-butanol, 2,3-dimethyl-1-butanol, 3,3-dimethyl-1-butanol, 2,3-dimethyl-2-butanol, 3,3-dimethyl-2-butanol, 2-ethyl-1-butanol, cyclobutanol, cyclopentanol, cyclohexanol, benzyl alcohol, and phenylethyl alcohol. These alcohols selected therefrom can be used singly or in combinations of two or more.

Among the (b) volatile substances, examples of the ketone include ketones having two alkyl groups having 1 to 4 carbon atoms such as acetone, methyl ethyl ketone, and methyl isobutyl ketone. These ketones can be used singly or two or more can be used in combination.

As the (b) volatile substance, one, or a combination of two or more selected from the group consisting of the alcohols and the ketones can be used, and water may be further contained.

The (b) volatile substance is more preferably one or more selected from the group consisting of ethanol, isopropyl alcohol, and n-butyl alcohol, further more preferably one or more selected from the group consisting of ethanol and isopropyl alcohol, and even more preferably ethanol. Additionally, the component (b) may contain 0.4 mass % or more and 10 mass % or less of water.

A content of the component (b) in the film-forming composition is preferably 45 mass % or more, more preferably 50 mass % or more, further more preferably 55 mass % or more, and even more preferably 60 mass % or more from a viewpoint of fiber formability. The content is preferably 95 mass % or less, more preferably 93 mass % or less, further more preferably 90 mass % or less, and even more preferably 88 mass % or less. From the same viewpoint, the content of the component (b) in the film-forming composition is preferably 45 mass % or more and 95 mass % or less, more preferably 50 mass % or more and 93 mass % or less, further more preferably 55 mass % or more and 90 mass % or less, and even more preferably 60 mass % or more and 88 mass % or less. When the component (b) is contained in this proportion in the film-forming composition, an intended coating film can be efficiently formed and a coating film composed of fibers can be formed stably. When the component (b) is contained in this proportion in the film-forming composition, the component (b) can be efficiently and sufficiently volatilized from the film-forming composition when the electrostatic spraying was implemented.

The non-volatile oil having a log S of −7 or more and less than 0 used as the component (c) is a component which imparts flexibility and elasticity to a coating film composed of fibers formed on the skin by electrostatic spraying to improve adhesion of the coating film to the skin and improve followability of the coating film to motion of the skin as well as improve durability of the coating film against moisture. The component (c) used in the present invention is a component which is a non-volatile oil and has a log S of −7 or more and less than 0.

Non-volatility refers to a property that a mass reduction rate obtained by spreading 1 g of an oil in a glass petri dish having a diameter of 48 mm, and leaving the resultant to stand still at 25° C. and normal pressure for 24 hours is 1% or less.

A log S is an index indicating solubility in water of a chemical substance, and “S” indicates a concentration (mol/L) of a saturated solution. The log S value used in the present invention is calculated with Chem Draw Professional 17.1, and a calculation module based on chemoinformatics platform MOSES of Molecular Networks is used in the calculation method. MOSES is developed, maintained and possessed by Molecular Networks GmbH (Germany, Erlangen).

The log S of the component (c) is preferably −6 or more, and more preferably −4 or more. The log S is preferably less than 0.

The component (c) is not particularly limited as long as it is generally used in the field of cosmetic products but it is possible to use, for example, one of, or a combination of two or more of non-volatile oils having a log S of −7 or more and less than 0 selected from the group consisting of a polyoxyalkylene glycol, a polyoxyalkylene alkyl ether, an ester oil, a silicone oil, a higher alcohol, a higher fatty acid, and a nonionic surfactant.

As the component (c), a non-volatile oil having a branch structure or an unsaturated structure is preferably used.

Examples of the polyoxyalkylene glycol include polypropylene glycol, polybutylene glycol, polyoxypropylene glyceryl ether, polyoxybutylene glyceryl ether, polyoxyalkylene glyceryl ether (those containing as the constituent unit of the oxyalkylene one or more alkylene oxides such as propylene oxide, and butylene oxide) and mixtures thereof. From the viewpoints of imparting flexibility and elasticity to the coating film composed of fibers formed on the skin by the electrostatic spraying to improve adhesion of the coating film to the skin and improve followability of the coating film to motion of the skin as well as improve durability of the coating film against moisture, the polyoxyalkylene glycol herein is preferably a poly-C3-C4 alkylene glycol, and more preferably a polypropylene glycol, and has an average molecular weight of preferably 3 000 or less, and more preferably 1 000 or less, and in particular, PPG-7 (log S: −0.9407), PPG-9 (log S: −1.472) and PPG-17 (log S: −3.596) are preferably used.

Examples of the polyoxyalkylene alkyl ether include polyoxyethylene lauryl ether, polyoxyethylene oleyl ether, polyoxypropylene lauryl ether, polyoxypropylene oleyl ether, polyoxybutylene lauryl ether, polyoxybutylene oleyl ether, polyoxyalkylene alkyl ether (which contains, as the constituent unit of the oxyalkylene, one or more alkylene oxides such as ethylene oxide, propylene oxide, and butylene oxide, and is an alkyl ether having an alkyl group having a straight chain or branched chain of about 1 to 20 carbon atoms), polyoxyethylene methyl glucoside, polyoxypropylene methyl glucoside, polyoxyalkylene methyl glucoside (which contains, as the constituent unit of the oxyalkylene, one or more alkylene oxides such as ethylene oxide, propylene oxide, and butylene oxide) and mixtures thereof. From the viewpoints of imparting flexibility and elasticity to the coating film composed of fibers formed on the skin by the electrostatic spraying to improve adhesion of the coating film to the skin and improve followability of the coating film to motion of the skin as well as improve durability of the coating film against moisture, polyoxyethylene lauryl ether is preferably used, examples include laureth-3 (log S: −3.377) and laureth-30 (log S: −0.7034), and laureth-3 (log S: −3.377) is preferably used.

Examples of the ester oil include sebacic acid diesters such as diethyl sebacate, isononanoic acid esters such as isononyl isononanoate (log S: −4.995) and isotridecyl isononanoate (log S: −6.349), ethylhexanoic acid esters such as cetyl ethylhexanoate (log S: −6.869), UV absorbers such as glycol stearate (log S: −4.8), glycol dilaurate (log S: −7.0), di-2-ethylhexyl sebacate (log S: −6.511), triethyl citrate (log S: −0.5133), triethyl acetylcitrate (log S: −1.402), tributyl acetylcitrate (log S: −3.432) and ethylhexyl methoxycinnamate (log S: −4.894), and alkyl benzoate. In particular, from the viewpoints of imparting flexibility and elasticity to the coating film composed of fibers formed on the skin by the electrostatic spraying to improve adhesion of the coating film to the skin and improve followability of the coating film to motion of the skin as well as improve durability of the coating film against moisture, di-2-ethylhexyl sebacate (log S: −6.511), triethyl citrate (log S: −0.5133), triethyl acetylcitrate (log S: −1.402), tributyl acetylcitrate (log S: −3.432) and ethylhexyl methoxycinnamate (log S: −4.894) are preferably used.

Examples of the silicone oil include chain silicones such as oxazoline-modified silicone, PEG-11 methyl ether dimethicone, PEG/PPG-20/22 butyl ether dimethicone, polyglyceryl-3 disiloxane dimethicone, polyglyceryl-3 disiloxane ethyl dimethicone, and lauryl polyglyceryl-3 disiloxane ethyl dimethicone.

Examples of the higher alcohol include saturated straight chain monovalent alcohols and unsaturated monovalent alcohols. Examples of the saturated straight chain monovalent alcohol include dodecanol (lauryl alcohol), tridodecanol, tetradodecanol (myristyl alcohol), pentadecanol, hexadecanol (cetyl alcohol), heptadecanol, octadecanol (stearyl alcohol), nonadecanol, icosanol (arachidyl alcohol), heneicosanol, docosanol (behenyl alcohol), tricosanol, tetracosanol (carnaubyl alcohol), pentacosanol, and hexacosanol (ceryl alcohol).

Examples of the unsaturated monovalent alcohol include oleyl alcohol and elaidyl alcohol.

Examples of the higher fatty acid include lauric acid, myristic acid, palmitic acid, stearic acid, oleic acid (log S: −4.754), palmitoleic acid (log S: −4.076), cis-6-hexadecenoic acid (log S: −4.076), linolic acid (log S: −4.627), linolenic acid (log S: −4.492), and ricinoleic acid (log S: −3.883). In particular, from the viewpoints of imparting flexibility and elasticity to the coating film composed of fibers formed on the skin by the electrostatic spraying to improve adhesion of the coating film to the skin and improve followability of the coating film to motion of the skin as well as improve durability of the coating film against moisture, an unsaturated fatty acid having 16 to 24 carbon atoms is preferably used, oleic acid (log S: −4.754), palmitoleic acid (log S: −4.076), cis-6-hexadecenoic acid (log S: −4.076), linolic acid (log S: −4.627), linolenic acid (log S: −4.492), and ricinoleic acid (log S: −3.883) are more preferably used, and oleic acid (log S: −4.754) is further more preferably used.

Examples of the nonionic surfactant include polyethylene glycol monofatty acid esters such as polyoxyethylene glycol monolaurate, and polyoxyethylene glycol monostearate, polypropylene glycol monofatty acid esters such as polyoxypropylene glycol monolaurate, and polyoxypropylene glycol monostearate, polybutylene glycol monofatty acid esters such as polyoxybutylene glycol monolaurate, and polyoxybutylene glycol monostearate, polyoxyalkylene glycol monofatty acid esters (those containing, as the constituent unit of the oxyalkylene, one or more alkylene oxides such as ethylene oxide, propylene oxide, and butylene oxide), and mixtures thereof, polyoxyalkylene glycol difatty acid esters (those containing, as the constituent unit of the oxyalkylene, one or more alkylene oxides such as ethylene oxide, propylene oxide, and butylene oxide, two fatty acids optionally being the same or different fatty acids), and mixtures thereof, fatty acid polyoxyethylene sorbitan, maltitol hydroxy aliphatic alkyl ether, alkylated polysaccharides, alkylglycosides, sucrose fatty acid esters, polyoxyethylene hydrogenated castor oil glyceryl, polyoxyethylene sorbit fatty acid esters, polyoxyethylene-polyoxypropylene block copolymers, tetrapolyoxyethylene-tetrapolyoxypropylene-ethylenediamine condensates, polyoxyethylene-beeswax-lanolin derivatives, alkanolamides, polyoxyethylene-propylene glycol fatty acid esters, polyoxyethylene-alkylamines, polyoxyethylene-fatty acid amides, alkylethoxydimethylamine oxides, trioleyl phosphates, and alkyl glyceryl ethers such as polyoxyethylene fatty acid glyceryls, isostearyl glyceryl ethers, isodecyl glyceryl ethers, and 2-ethylhexyl glyceryl ethers. In particular, from the viewpoints of imparting flexibility and elasticity to the coating film composed of fibers formed on the skin by the electrostatic spraying to improve adhesion of the coating film to the skin and improve followability of the coating film to motion of the skin as well as improve durability of the coating film against moisture, fatty acid polyoxyethylene sorbitan is preferably used, and polyoxyethylene (20) sorbitan monostearate (log S: −2.883) is preferably used.

Among these components (c), from the viewpoints of the adhesion of the coating film to the skin, the followability of the coating film to motion of the skin, and the durability of the coating film against moisture, it is preferable to use one or more selected from the group consisting of a poly-C3-C4 alkylene glycol, a polyoxyalkylene alkyl ether, an ester oil, a higher fatty acid, and a nonionic surfactant, and it is more preferable to use a polyoxyalkylene alkyl ether, a higher fatty acid, or a nonionic surfactant.

When the polymer having film-forming ability as the component (a) is a polyvinyl butyral resin or the like, preferable examples of the component (c) include the following: The non-volatile oil serving as the component (c) is preferably a compound easily interactable with a hydroxy group, an ester or an acetal moiety present in the structure of the polyvinyl butyral resin, and specific examples include a poly-C3-C4 alkylene glycol, a polyoxyalkylene alkyl ether, an ester oil, a silicone oil, a nonionic (non-ionic) surfactant, and a higher fatty acid. From the viewpoints of a favorable elastic modulus, and improvement of the adhesion, the followability to motion of the skin, the durability against moisture, and a feel of the coating film, it is preferable to contain a polypropylene glycol, an ester oil, a nonionic surfactant, a polyoxyalkylene alkyl ether, or a higher fatty acid, and a polyoxyalkylene alkyl ether, a higher fatty acid, or a nonionic surfactant is more preferably used. One or more selected from these can be used.

A content of the component (c) in the film-forming composition is preferably 0.1 mass % or more, preferably 0.5 mass % or more, preferably 1 mass % or more, preferably 2 mass % or more, more preferably 3 mass % or more, further more preferably 3.5 mass % or more, and even more preferably 4 mass % or more. The content is preferably 30 mass % or less, more preferably 25 mass % or less, further more preferably 20 mass % or less, and further more preferably 18 mass % or less, further more preferably 15 mass % or less, further more preferably 10 mass % or less, and even more preferably 8 mass % or less. The content of the component (c) in the film-forming composition is preferably 2 mass % or more and 30 mass % or less, more preferably 3 mass % or more and 25 mass % or less, further more preferably 3 mass % or more and 20 mass % or less, further more preferably 3 mass % or more and 18 mass % or less, further more preferably 3 mass % or more and 10 mass % or less, further more preferably 3.5 mass % or more and 10 mass % or less, and even more preferably 4 mass % or more and 8 mass % or less. When the content of the component (c) is in this range, flexibility and a favorable elastic modulus are imparted to the coating film composed of fibers formed on the skin by the electrostatic spraying, the adhesion to the skin and moisture durability of the coating film are improved, the followability of the coating film to motion of the skin is improved, and in addition, the skin compatibility of the coating film is improved.

A content mass ratio of the component (a) to the component (c) in the film-forming composition, ((a)/(c)), is, from the viewpoints of the flexibility, the favorable elastic modulus, the skin compatibility, the adhesion and the durability against moisture of the coating film composed of fibers formed by the electrostatic spraying, preferably 0.15 or more, more preferably 0.25 or more, further more preferably 0.5 or more, further more preferably 1 or more, and even more preferably 1.5 or more. Additionally, it is preferably 300 or less, more preferably 50 or less, further more preferably 15 or less, further more preferably 13 or less, further more preferably 10 or less, further more preferably 8 or less, and even more preferably 6 mass % or less. Such (a)/(c) is preferably 0.15 or more and 300 or less, more preferably 0.25 or more and 50 or less, further more preferably 0.5 or more and 50 or less, further more preferably 0.5 or more and 15 or less, and even more preferably 1 or more and 15 or less. Additionally, it is preferably 1 or more and 13 or less, more preferably 1 or more and 10 or less, and further more preferably 1.5 or more and 8 or less.

A content mass ratio of the component (b) to the component (c) in the film-forming composition, ((b)/(c)), is, from the viewpoints of the flexibility, the favorable elastic modulus, the skin compatibility, the adhesion and the durability against moisture of the coating film composed of fibers formed by the electrostatic spraying, preferably 3 or more, more preferably 5 or more, further more preferably 7 or more, and even more preferably 9 or more. Additionally, it is preferably 200 or less, more preferably 100 or less, further more preferably 50 or less, further more preferably 30 or less, and even more preferably 22 or less. Such a content mass ratio ((b)/(c)) is preferably 3 or more and 200 or less, more preferably 5 or more and 100 or less, further more preferably 7 or more and 50 or less, further more preferably 7 or more and 30 or less, and even more preferably 9 or more and 22 or less.

A content mass ratio of the component (a) to the component (b) in the film-forming composition, ((a)/(b)), is, from a viewpoint of efficiently forming a desired coating film, from a viewpoint of stably forming the coating film composed of fibers, and from a viewpoint of efficiently and sufficiently volatilizing the component (b) from the film-forming composition in implementing the electrostatic spraying, preferably 0.03 or more, more preferably 0.05 or more, further more preferably 0.1 or more, and even more preferably 0.12 or more. Additionally, from the same viewpoints, it is preferably 0.6 or less, more preferably 0.45 or less, further more preferably 0.35 or less, further more preferably 0.3 or less, further more preferably 0.25 or less, further more preferably 0.2 or less, and even more preferably 0.18 or less. Such (a)/(b) is preferably 0.03 or more and 0.6 or less, more preferably 0.05 or more and 0.45 or less, further more preferably 0.1 or more and 0.35 or less, further more preferably 0.1 or more and 0.3 or less, and even more preferably 0.12 or more and 0.25 or less.

The film-forming composition may contain, in addition to the above components, a conductivity controlling agent, an oil agent other than the components (c), a coloring pigment, an extender pigment, a dye, a fragrance, a repellent, an antioxidant, a stabilizer, a preservative, vitamins, and water. The conductivity controlling agent is, from a viewpoint of the conductivity enhancement, preferably an alkali metal salt or an ammonium salt, more preferably an ionic surfactant, and further more preferably one or more selected from the group consisting of a cationic surfactant and an anionic surfactant.

A content of the conductivity controlling agent in the film-forming composition is not limited as long as an amount achieves the conductivity of the composition within the above ranges but, from a viewpoint of stably forming a coating film and a viewpoint of preventing a conductivity from excessively increasing, preferably 0.01 mass % or more and 10 mass % or less, more preferably 0.05 mass % or more, and further more preferably 0.1 mass % or more, and more preferably 8 mass % or less, further more preferably 6 mass % or less, further more preferably 2.5 mass % or less, and even more preferably 2 mass % or less.

A viscosity of the film-forming composition at 25° C. is, from a viewpoint of stably forming a coating film composed of fibers on the skin and a viewpoint of spinning property when electrostatically spraying, drying of fibers, and thinning diameters of fibers, preferably 2 mPa·s or more, more preferably 5 mPa·s or more, further more preferably 10 mPa·s or more, further more preferably 30 mPa·s or more, further more preferably 50 mPa·s or more, and even more preferably 80 mPa·s or more. Additionally, it is preferably 3 000 mPa·s or less, more preferably 2 000 mPa·s or less, further more preferably 1 500 mPa·s or less, further more preferably 1 000 mPa·s or less, further more preferably 800 mPa·s or less, and even more preferably 500 mPa·s or less. A viscosity of the film-forming composition ranges preferably 2 mPa·s or more and 3 000 mPa·s or less, more preferably 5 mPa·s or more and 2 000 mPa·s or less, further more preferably 10 mPa·s or more and 1 500 mPa·s or less, further more preferably 30 mPa·s or more and 1 000 mPa·s or less, further more preferably 50 mPa·s or more and 800 mPa·s or less, and even more preferably 80 mPa·s or more and 500 mPa·s or less. A viscosity of the film-forming composition is measured using a B-type viscometer at 25° C. For the B-type viscometer, for example, a B-type viscometer (TVB-10M) manufactured by Toki Sangyo Co., Ltd. can be used. The measurement condition in such a case has a measurement temperature of 25° C. The measurement temperature herein refers to a temperature of the film-forming composition. Type of a rotor and a rotation speed of the rotor are selected in accordance with specifications of a measurement apparatus to be used in accordance with a viscosity of the composition for forming a coating composition. For example, when the above B-type viscometer (TVB-10M) manufactured by Toki Sangyo Co., Ltd. is used, the measurement can be achieved using an M2 rotor at 6 rpm when a viscosity of the composition for a coating film is 2 500 mPa·s or more, an M2 rotor at 12 rpm when such a viscosity is 1 000 mPa·s or more and less than 2 500 mPa·s, an M2 rotor at 30 rpm when such a viscosity is 500 mPa·s or more and less than 1 000 mPa·s, an M2 rotor at 60 rpm when such a viscosity is 100 mPa·s or more and less than 500 mPa·s, and an M1 rotor at 60 rpm when such a viscosity is less than 100 mPa·s. Additionally, instructions for use of the above B-type viscometer (TVB-10M) manufactured by Toki Sangyo Co., Ltd. also include measurement conditions other than the above measurement conditions and a viscosity can also be measured under other measurement conditions in accordance with a viscosity of the film-forming composition.

Subsequently described is a method for forming a coating film composed of fibers directly formed on the skin by electrostatic spraying using the film-forming composition of the present invention.

The film-forming composition may be sprayed directly to a site at which a coating film is intended to be formed on the human skin by the electrostatic spraying method. The electrostatic spraying method includes a step for electrostatically spraying the film-forming composition to the skin using an electrostatic spraying apparatus. The electrostatic spraying apparatus basically has a container for accommodating the above composition, a nozzle for discharging the composition, a feed apparatus for feeding the composition accommodated in the container to the nozzle, and a power supply for applying a voltage to the nozzle.

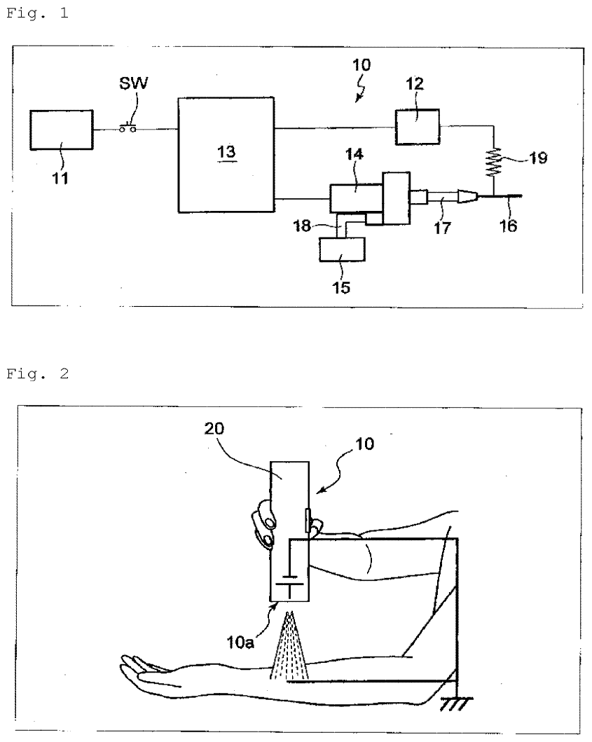

FIG. 1 is a diagram showing a structure of the electrostatic spraying apparatus preferably used in the present invention. An electrostatic spraying apparatus 10 shown in the same figure is equipped with a low-voltage power supply 11. The low-voltage power supply 11 is for generating several volts to ten and some volts.

The low-voltage power supply 11 is preferably composed of one or more batteries for the purpose of increasing the portability of the electrostatic spraying apparatus 10.

Additionally, batteries, when used as the low-voltage power supply 11, can be easily replaceable as needed, hence advantageous. Instead of batteries, an AC adapter can also be used as the low-voltage power supply 11.

The electrostatic spraying apparatus 10 is also equipped with a high-voltage power supply 12. The high-voltage power supply 12 is connected to the low-voltage power supply 11 and equipped with an electric circuit (not shown) to boost the voltage generated at the low-voltage power supply 11 to a high voltage. A voltage boost electric circuit is generally made up of a transformer, a capacitor, and a semiconductor element.

The electrostatic spraying apparatus 10 is further equipped with an auxiliary electric circuit 13. The auxiliary electric circuit 13 is interposed between the low-voltage power supply 11 and the high-voltage power supply 12 described above and functions to adjust a voltage of the low-voltage power supply 11 thereby allowing the high-voltage power supply 12 to operate stably. The auxiliary electric circuit 13 has a function of controlling the rotation speed of a motor equipped by a micro gear pump 14 to be described later. Controlling the rotation speed of the motor serves to control a feed amount of the film-forming composition to the micro gear pump 14 from a container 15 for the film-forming composition. A switch SW is attached between the auxiliary electric circuit 13 and the low-voltage power supply 11 and the electrostatic spraying apparatus 10 can start/stop by switching ON-OFF the switch SW.

The electrostatic spraying apparatus 10 is further equipped with a nozzle 16. The nozzle 16 is composed of various conductors such as a metal to begin with and non-conductors such as plastic, rubber, and ceramic, and has a shape which can discharge the film-forming composition from the tip thereof. Inside the nozzle 16, a microspace through which the film-forming composition passes is formed longitudinally along with the nozzle 16. A cross-sectional size of this microspace is preferably 100 μm or more and 1 000 μm or less when expressed in the diameter.

The nozzle 16 communicates with the micro gear pump 14 via a pipeline 17. The pipeline 17 may be a conductor or a non-conductor. Additionally, the nozzle 16 is electrically connected to the high-voltage power supply 12. This enables the application of a high-voltage to the nozzle 16. In this case, the nozzle 16 and the high-voltage power supply 12 are electrically connected via an electric current limiting resistor 19 to prevent an excess electric current from flowing when a human body directly touches the nozzle 16.

The micro gear pump 14 communicating with the nozzle 16 via the pipeline 17 functions as a feed apparatus for feeding the film-forming composition accommodated in the container 15 to the nozzle 16. The micro gear pump 14 receives a feed of power supply from the low-voltage power supply 11 and operates. The micro gear pump 14 is configured in such a way as to feed a predetermined amount of the film-forming composition to the nozzle 16 in response to the control by the auxiliary electric circuit 13.

The container 15 is connected to the micro gear pump 14 via a flexible pipeline 18. In the container 15, the film-forming composition is accommodated. The container 15 is preferably an exchangeable cartridge type. It is also possible to employ a configuration using a piston pump instead of the micro gear pump 14 in such a way as to feed a predetermined amount of the film-forming composition to the nozzle 16 in response to the control by the auxiliary electric circuit 13.

The electrostatic spraying apparatus 10 having the above structure can be used as shown in, for example, FIG. 2. FIG. 2 shows a hand-held electrostatic spraying apparatus 10 having a size holdable by one hand. The electrostatic spraying apparatus 10 shown in the same figure accommodates all the members of the structural diagram shown in FIG. 1 inside a cylindrical housing 20. The nozzle (not shown) is arranged at an end 10a of a longitudinal direction of the housing 20. The nozzle is arranged in the housing 20 with a coming-out direction of the composition in line with the longitudinal direction of the housing 20 so that it protrudes toward the skin side. When the nozzle tip is arranged in such a way as to protrude toward the skin along with the longitudinal direction of the housing 20, the film-forming composition is less likely to attach to the housing thereby stably forming a coating film.

In operating the electrostatic spraying apparatus 10, a user, i.e., a person who intends to form a coating film on a site at which the coating film is formed on the skin by electrostatic spraying, holds the apparatus 10 with a hand and turns the end 10a of the apparatus 10 at which the nozzle (not shown) is arranged toward an application site to which electrostatic spraying is implemented. FIG. 2 shows a state in which an end 10a of the electrostatic spraying apparatus 10 is turned toward the inner side of the user's forearm. In this state, the apparatus 10 is switched ON to implement the electrostatic spraying. When the apparatus 10 is turned on, an electric field is generated between the nozzle and the skin.

In the embodiment shown in FIG. 2, a positive high voltage is applied to the nozzle thereby making the skin a negative electrode. When the electric field is generated between the nozzle and the skin, the film-forming composition at the nozzle tip section is polarized due to electrostatic induction and forms a corn shape at the tip part, and droplets of the charged film-forming composition is discharged into the air from the corn tip toward the skin along with the electric field.

As the component (b), a solvent, evaporates from the charged film-forming composition discharged into the air, the film-forming composition has an excessed charge density on the surface, proceeds in the air by Coulomb repulsion while repeatedly broken down, and reaches the skin. In this instance, when a viscosity of the film-forming composition is suitably adjusted, the volatile substance, a solvent, is caused to volatilize from droplets while the composition is discharged into the air, and the polymer having film-forming ability, a solute, of the component (a) forms fibers as elongated by a potential difference while solidified, whereby the formed fibers are caused to deposit at an application site. For example, when a viscosity of the film-forming composition is increased, the composition is likely to deposit at an application site in the form of fibers.

Herewith, a porous coating film composed of fibrous deposits is formed on the surface of an application site.

Such a porous coating film composed of fibrous deposits can also be formed by adjusting the distance between the nozzle and the skin or a voltage to be applied to the nozzle.

A high potential difference is being generated between the nozzle and the skin while the electrostatic spraying is implemented. However, the impedance is so significant that an electric current flowing through the human body is extremely small. For example, the present inventor confirmed that, for example, an electric current flowing through the human body while the electrostatic spraying is implemented is some digits smaller than an electric current flowing through the human body by static electricity generated in daily life.

When fibrous deposits are formed by the electrostatic spraying method, a thickness of fibers when expressed by a diameter equivalent to a circle is preferably 50 nm or more, more preferably 100 nm or more, and further more preferably 200 nm or more.

Additionally, the thickness is preferably 3 000 nm or less, more preferably 1 500 nm or less, and further more preferably 1 000 nm or less.

The thickness of fibers can be measured by, for example, magnifying the fibers 10 000 times to observe using a scanning electron microscopic (SEM), excluding defects (fiber masses, fiber overlaps, and droplets) from the two-dimensional images, randomly selecting 10 fibers, drawing a line perpendicular to the longitudinal direction of the fibers, and directly reading a diameter of the fibers.

The fiber in the present invention is preferably a continuous fiber and preferably has a length of at least 100 times or more a thickness of the fiber. For example, a formed coating film preferably contains a fiber containing the component (a) and having a length of preferably 10 μm or more, more preferably 50 μm or more, and further more preferably 100 μm or more. In the present specification, a fiber having a length of 100 times or more a thickness of the fiber is defined as the “continuous fiber.” The cross-sectional shape of the fiber is preferably circle or oval, and the fiber thickness refers to the diameter in the case of circle and a length of the major axis in the case of oval. The coating film produced by the electrostatic spraying method is preferably porous non-continuous coating film composed of one or more continuous fibrous deposits.

According to the electrostatic spraying method, the film-forming composition is charged and sprayed and affected by the moisture in the air when a humidity is high but can be less likely affected when a conductivity controlling agent is contained.

In the method for producing the film-forming composition, one may stir a mixed solution containing all components but it is preferable to have the step 1 for stirring Mixed solution 1 containing the components other than the component (a) and subsequently the step 2 for adding the component (a) followed by stirring and mixing. These step 1 and step 2 are preferably implemented at normal temperature of 10° C. to 30° C.

A distance between the nozzle and the skin is, in accordance with a voltage to be applied to the nozzle through, preferably 10 mm or more, more preferably 20 mm or more, and further more preferably 40 mm or more. Additionally, it is preferably 160 mm or less, more preferably 140 mm or less, and further more preferably 120 mm or less. A distance between the nozzle and the skin within this range can enhance the formability of a coating film. A distance between the nozzle and the skin can be measured by a commonly used non-contact sensor.

A basis weight of the coating film is, regardless of the coating film formed by the electrostatic spraying method being porous or not, preferably 0.10 g/m² or more, and more preferably 1.0 g/m² or more. Additionally, it is preferably 50 g/m² or less, and more preferably 40 g/m² or less. A basis weight of the formed coating film is preferably 0.10 g/m² or more and 50 g/m² or less, and more preferably 1.0 g/m² or more and 40 g/m² or less.

When the basis weight of the coating film is thus set, the skin compatibility of the coating film, the adhesion, and the followability of the coating film to the skin, and the durability against moisture can be improved.

An elastic modulus of the coating film formed on the skin using the film-forming composition of the present invention is, from the viewpoints of the adhesion of the coating film and the followability to motion of the skin, preferably 1×10⁷ N/m² or less, more preferably 1×10⁴ N/m² or more and 9×10⁶ N/m² or less, further more preferably 1×10⁴ N/m² or less and 8×10⁶ N/m² or less, and even more preferably 1×10⁴ N/m² or more and 7×10⁶ N/m² or less. The elastic modulus can be adjusted mainly in accordance with the type and the content of the component (c). Herein, the elastic modulus is a numerical value measured at 25° C. with a tensile tester. As the tensile tester, Tensilon UTC-100W manufactured by Orientec Co., Ltd. can be used. A coating film is formed by implementing the electrostatic spraying in a center portion of an aluminum foil (manufactured by As One Corporation) of 10 cm×10 cm for 7.5. A notch is made in the aluminum foil in a vertical direction to a short side (2.5 cm) of a hole, and then, the resultant is pulled in a direction parallel to the short side of the hole at a tensile speed of 500 mm/min to measure a stress-strain curve. An initial slope of the stress-strain curve is calculated, and an elastic modulus at a width of 7.5 cm and a length of 2.5 cm is calculated assuming that a thickness has a constant value of 20 μm.

In the present invention, a skincare cosmetic or a makeup cosmetic may be applied to the skin before or after the electrostatic spraying step for forming a coating film on the skin by electrostatic spraying using the film-forming composition of the present invention described above. When the electrostatic spraying is thus implemented before or after a cosmetic is applied, the cosmetic present on the skin can be covered with the coating film so as to remarkably improve the compatibility with the skin of the cosmetic, and improve the durability against moisture, and thus, the cosmetic can be retained on the skin stably for a long period of time.

The skincare cosmetic used herein includes skin lotions, milky lotions, creams, serums (whitening, anti-wrinkle, etc.), all-in-one cosmetics, UV care cosmetics, BB creams, oils, oil gels, and lotions, and the makeup cosmetic includes makeup bases, foundations, concealers, cheek blushers, eye shadows, mascaras, eye liners, eyebrows, overcoat agents, and lipsticks.

Examples of the application means of the above skincare cosmetics or makeup cosmetics to the skin other than the electrostatic spraying include application by hands and/or fingers, application using a nonwoven cloth such as cotton, application using a sponge, spraying using a usual spray, spraying mist, steaming, dripping, and sprinkling.

In reference with the embodiments described above, the present invention further discloses the following compositions and methods.

<1> A film-forming composition, for forming a coating film composed of fibers directly on the skin by electrostatic spraying, wherein the film-forming composition comprises the following components (a), (b) and (c), and the coating film formed from the film-forming composition has an elastic modulus of 1×10⁴ N/m² or more and 9×10⁶ N/m² or less:

(a) a polymer having film-forming ability;

(b) one or more volatile substances selected from the group consisting of an alcohol and a ketone; and

(c) 2 mass % or more and 30 mass % or less of a non-volatile oil having a log S of −7 or more and less than 0.

<2> The film-forming composition according to <1>, wherein the component (a) is preferably one or more selected from water-insoluble polymers having film-forming ability.
<3> The film-forming composition according to <1> or <2>, wherein the component (a) is a water-insoluble polymer having film-forming ability, and preferably one or more selected from the group consisting of a completely saponified polyvinyl alcohol insolubilizable after forming a coating film, a partially saponified polyvinyl alcohol crosslinkable after forming a coating film when used in combination with a crosslinking agent, an oxazoline-modified silicone such as a poly(N-propanoylethyleneimine)graft-dimethylsiloxane/γ-aminopropylmethylsiloxane copolymer, a polyvinyl acetal diethylamino acetate, Zein (main component of corn protein), a polyester, a polylactic acid (PLA), an acrylic resin such as a polyacrylonitrile resin, and a polymethacrylic acid resin, and a polystyrene resin, a polyvinyl butyral resin, an alkyl acetalized polyvinyl alcohol, a polyethylene terephthalate resin, a polybutylene terephthalate resin, a polyurethane resin, a polyamide resin, a polyimide resin, and a polyamideimide resin, and further more preferably one or more selected from the group consisting of a completely saponified polyvinyl alcohol insolubilizable after forming a coating film, a partially saponified polyvinyl alcohol crosslinkable after forming a coating film when used in combination with a crosslinking agent, a polyvinyl butyral resin, an alkyl acetalized polyvinyl alcohol, a polyurethane resin, an oxazoline-modified silicone, a polyvinyl acetal diethylamino acetate, and Zein.
<4> The film-forming composition according to any one of <1> to <3>, wherein a content of the component (a) is preferably 4 mass % or more, more preferably 5 mass % or more, further more preferably 6 mass % or more, and even more preferably 8 mass % or more, is preferably 35 mass % or less, more preferably 30 mass % or less, further more preferably 25 mass % or less, and even more preferably 20 mass % or less, and is preferably 4 mass % or more and 30 mass % or less, more preferably 5 mass % or more and 25 mass % or less, further more preferably 6 mass % or more and 25 mass % or less, and even more preferably 8 mass % or more and 20 mass % or less.
<5> The film-forming composition according to any one of <1> to <4>, wherein a content of the component (a) is 4 mass % or more and 30 mass % or less.
<6> The film-forming composition according to any one of <1> to <4>, wherein a content of the component (a) is 4 mass % or more and 25 mass % or less.
<7> The film-forming composition according to any one of <1> to <6>, wherein the alcohol of the component (b) is preferably one or more selected from the group consisting of a monovalent chain fatty alcohol, a monovalent cyclic fatty acid, and a monovalent aromatic alcohol, preferably one or more selected from the group consisting of a straight chain or branched chain monovalent chain fatty alcohol having 1 to 6 carbon atoms, a monovalent cyclic fatty alcohol having 4 to 6 carbon atoms, benzyl alcohol, and phenyl ethyl alcohol, further more preferably one or more selected from the group consisting of ethanol, isopropyl alcohol, n-butyl alcohol, phenyl ethyl alcohol, n-propanol, and n-pentanol.
<8> The film-forming composition according to any one of <1> to <7>, wherein the ketone of the component (b) is preferably a ketone having two alkyl groups having 1 to 4 carbon atoms, and further more preferably one or more selected from the group consisting of acetone, methyl ethyl ketone, and methyl isobutyl ketone.
<9> The film-forming composition according to any one of <1> to <8>, wherein the component (b) is preferably one or more selected from the group consisting of ethanol, isopropyl alcohol and n-butyl alcohol, more preferably one or more selected from the group consisting of ethanol and isopropyl alcohol, further more preferably ethanol, and may further comprise water.
<10> The film-forming composition according to any one of <1> to <9>, wherein a content of the component (b) is preferably 45 mass % or more, more preferably 50 mass % or more, further more preferably 55 mass % or more, and even more preferably 60 mass % or more, and preferably 95 mass % or less, more preferably 93 mass % or less, further more preferably 90 mass % or less, and even more preferably 88 mass % or less, and preferably 45 mass % or more and 95 mass % or less, more preferably 50 mass % or more and 93 mass % or less, further more preferably 50 mass % or more and 90 mass % or less, and even more preferably 50 mass % or more and 88 mass % or less.
<11> The film-forming composition according to any one of <1> to <10>, wherein a content of the component (b) is 50 mass % or more and 95 mass % or less.
<12> The film-forming composition according to any one of <1> to <10>, wherein a content of the component (b) is 50 mass % or more and 93 mass % or less.
<13> The film-forming composition according to any one of <1> to <12>, wherein the component (c) is preferably one or more non-volatile oils having a log S of −7 or more and less than 0 selected from the group consisting of a polyoxyalkylene glycol, a polyoxyalkylene alkyl ether, an ester oil, a silicone oil, a higher alcohol, a higher fatty acid and a nonionic surfactant, and more preferably one or more non-volatile oils having a log S of −7 or more and less than 0 selected from the group consisting of a poly-C3-C4 alkylene glycol, a polyoxyalkylene alkyl ether, an ester oil, a higher fatty acid, and a nonionic surfactant.
<14> The film-forming composition according to any one of <1> to <13>, wherein the component (c) is preferably a compound easily interactable with a hydroxy group, an ester or an acetal moiety present in the structure of a polyvinyl butyral resin, more preferably one or more selected from the group consisting of a poly-C3-C4 alkylene glycol, a polyoxyalkylene alkyl ether, an ester oil, a silicone oil, a nonionic (non-ionic) surfactant, a higher fatty acid, and a UV absorber, and further more preferably one or more selected from the group consisting of a polypropylene glycol, an ester oil, a nonionic surfactant, a polyoxyalkylene alkyl ether, and a higher fatty acid.
<15> The film-forming composition according to any one of <1> to <14>, wherein a content of the component (c) is preferably 0.1 mass % or more, more preferably 0.5 mass % or more, further more preferably 1 mass % or more, further more preferably 2 mass % or more, further more preferably 3 mass % or more, further more preferably 3.5 mass %, and even more preferably 4 mass % or more; and is preferably 30 mass % or less, more preferably 25 mass % or less, further more preferably 20 mass % or less, further more preferably 18 mass % or less, further more preferably 15 mass % or less, and further more preferably 10 mass % or less, and even more preferably 8 mass % or less, and preferably 2 mass % or more and 30 mass % or less, more preferably 3 mass % or more and 25 mass % or less, further more preferably 3 mass % or more and 20 mass % or less, further more preferably 3 mass % or more and 20 mass % or less, further more preferably 3 mass % or more and 18 mass % or less, further more preferably 3 mass % or more and 10 mass % or less, further more preferably 3.5 mass % or more and 10 mass % or less, and even more preferably 4 mass % or more and 8 mass % or less.
<16> The film-forming composition according to any one of <1> to <15>, wherein a content of the component (c) is 2 mass % or more and 25 mass % or less.
<17> The film-forming composition according to any one of <1> to <15>, wherein a content of the component (c) is 3 mass % or more and 20 mass % or less.
<18> The film-forming composition according to any one of <1> to <17>, wherein a content mass ratio of the component (a) to the component (c), ((a)/(c)), is preferably 0.15 or more, more preferably 0.25 or more, further more preferably 0.5 or more, further more preferably 1 or more, and even more preferably 1.5 or more, and preferably 300 or less, more preferably 50 or less, further more preferably 15 or less, further more preferably 13 or less, further more preferably 10 or less, further more preferably 8 or less, and even more preferably 6 or less, and preferably 0.15 or more and 300 or less, more preferably 0.25 or more and 50 or less, further more preferably 0.5 or more and 50 or less, further more preferably 0.5 or more and 15 or less, further more preferably 1 or more and 15 or less, further more preferably 1 or more and 13 or less, further more preferably 1 or more and 10 or less, and even more preferably 1.5 or more and 8 or less.
<19> The film-forming composition according to any one of <1> to <18>, wherein a content mass ratio of the component (a) to the component (c), ((a)/(c)), is 0.15 or more and 300 or less.
<20> The film-forming composition according to any one of <1> to <19>, wherein a content mass ratio of the component (a) to the component (c), ((a)/(c)), is 0.25 or more and 50 or less.
<21> The film-forming composition according to any one of <1> to <20>, wherein a content mass ratio of the component (b) to the component (c), ((b)/(c)), is preferably 3 or more, more preferably 5 or more, more preferably 7 or more, and even more preferably 9 or more; and is preferably 200 or less, more preferably 100 or less, more preferably 50 or less, more preferably 30 or less, and even more preferably 22 or less, and preferably 3 or more and 200 or less, more preferably 5 or more and 100 or less, further more preferably 7 or more and 50 or less, further more preferably 7 or more and 30 or less, and even more preferably 9 or more and 22 or less.
<22> The film-forming composition according to any one of <1> to <21>, wherein a content mass ratio of the component (b) to the component (c), ((b)/(c)), is preferably 3 or more and 200 or less.
<23> The film-forming composition according to any one of <1> to <22>, wherein a content mass ratio of the component (a) to the component (b), ((a)/(b)), is preferably 0.03 or more, more preferably 0.05 or more, further more preferably 0.1 or more, and even more preferably 0.12 or more, and preferably 0.6 or less, more preferably 0.45 or less, further more preferably 0.35 or less, further more preferably 0.3 or less, further more preferably 0.25 or less, further more preferably 0.2 or less, and even more preferably 0.18 or less, and preferably 0.03 or more and 0.6 or less, more preferably 0.05 or more and 0.45 or less, further more preferably 0.1 or more and 0.35 or less, further more preferably 0.1 or more and 0.3 or less, and even more preferably 0.12 or more and 0.25 or less.
<24> The film-forming composition according to any one of <1> to <23>, wherein a content mass ratio of the component (a) to the component (b), ((a)/(b)), is 0.03 or more and 0.6 or less.
<25> The film-forming composition according to any one of <1> to <24>, wherein a content mass ratio of the component (a) to the component (b), ((a)/(b)), is 0.05 or more and 0.45 or less.
<26> The film-forming composition according to any one of <1> to <25> comprising components selected from the group consisting of a conductivity controlling agent, an oil agent other than the component (c), a coloring pigment, an extender pigment, a dye, a fragrance, a repellent, an antioxidant, a stabilizer, a preservative, a vitamin, and water.
<27> The film-forming composition according to <26>, wherein the conductivity controlling agent is preferably a component achieving a conductivity of the film-forming composition at 25° C. of 10 μS/cm or more and 300 μS/cm or less.
<28> The film-forming composition according to <26> or <27>, wherein the conductivity controlling agent is preferably an alkali metal salt or an ammonium salt, more preferably an ionic surfactant, and further more preferably one or more selected from the group consisting of a cationic surfactant and an anionic surfactant.
<29> The film-forming composition according to any one of <26> to <28>, wherein the conductivity controlling agent is one or more selected from the group consisting of a quaternary ammonium salt and an acyl amino acid salt.
<30> The film-forming composition according to any one of <26> to <29>, wherein a content of the conductivity controlling agent is preferably 0.01 mass % or more, more preferably 0.05 mass % or more, and further more preferably 0.1 mass % or more, and preferably 10 mass % or less, more preferably 8 mass % or less, further more preferably 6 mass % or less, further more preferably 2.5 mass % or less, and even more preferably 2 mass % or less, and preferably 0.01 mass % or more and 10 mass % or less, more preferably 0.05 mass % or more and 8 mass % or less, further more preferably 0.1 mass % or more and 6 mass % or less, further more preferably 0.1 mass % or more and 2.5 mass % or less, and even more preferably 0.1 mass % or more and 2 mass % or less.
<31> The film-forming composition according to any one of <1> to <30>, wherein a viscosity of the film-forming composition at 25° C. is preferably 2 mPa·s or more, more preferably 5 mPa·s or more, further more preferably 10 mPa·s or more, further more preferably 30 mPa·s or more, further more preferably 50 mPa·s or more, and even more preferably 80 mPa·s or more, and preferably 3 000 mPa·s or less, more preferably 2 000 mPa·s or less, further more preferably 1 500 mPa·s or less, further more preferably 1 000 mPa·s or less, further more preferably 800 mPa·s or less, and even more preferably 500 mPa·s or less, and preferably 2 mPa·s or more and 3 000 mPa·s or less, more preferably 5 mPa·s or more and 2 000 mPa·s or less, further more preferably 10 mPa·s or more and 1 500 mPa·s or less, further more preferably 30 mPa·s or more and 1 000 mPa·s or less, further more preferably 50 mPa·s or more and 800 mPa·s or less, and even more preferably 80 mPa·s or more and 500 mPa·s or less.
<32> The film-forming composition according to any one of <1> to <31>, wherein an obtained coating film has an elastic modulus of preferably 1×10⁴ N/m² or more and 8×10⁶ N/m² or less, and more preferably 1×10⁴ N/m² or less and 7×10⁶ N/m² or less.
<33> The film-forming composition according to any one of <1> to <32>, used in combination with a cosmetic applied to the skin by a device other than an electrostatic spray.
<34> The film-forming composition according to <33>, wherein the cosmetic is preferably a skincare cosmetic or a makeup cosmetic.
<35> A film-forming composition, for forming a coating film composed of fibers directly on the skin by electrostatic spraying, wherein the composition comprises the following components (a), (b) and (c), and the coating film formed from the film-forming composition has an elastic modulus of 1×10⁴ N/m² or more and 9×10⁶ N/m² or less:

(a) 4 mass % or more and 35 mass % or less of a polymer having film-forming ability;

(b) 45 mass % or more and 95 mass % or less of one or more volatile substances selected from the group consisting of an alcohol and a ketone; and

(c) 2 mass % or more and 30 mass % or less of a non-volatile oil having a log S of −7 or more and less than 0.

<36> A method for producing a coating film on a surface of a coating film formation subject, wherein the method comprises an electrostatically spraying step for forming a coating film composed of deposits comprising fibers by directly electrostatically spraying a composition on the coating film formation subject, the composition comprises the following components (a), (b) and (c), and the coating film formed from the composition has an elastic modulus of 1×10⁴ N/m² or more and 9×10⁶ N/m² or less:

(a) 4 mass % or more and 35 mass % or less of a polymer having film-forming ability;

(b) 45 mass % or more and 95 mass % or less of one or more volatile substances selected from the group consisting of an alcohol and a ketone; and

(c) 2 mass % or more and 30 mass % or less of a non-volatile oil having a log S of −7 or more and less than 0.

<37> A cosmetic comprising a film-forming composition for forming a coating film composed of fibers directly on the skin by electrostatic spraying, wherein the composition comprises the following components (a), (b) and (c), and a coating film formed from the film-forming composition has an elastic modulus of 1×10⁴ N/m² or more and 9×10⁶ N/m² or less:

(a) 4 mass % or more and 35 mass % or less of a polymer having film-forming ability;

(b) 45 mass % or more and 95 mass % or less of one or more volatile substances selected from the group consisting of an alcohol and a ketone; and

(c) 2 mass % or more and 30 mass % or less of a non-volatile oil having a log S of −7 or more and less than 0.

### EXAMPLES

Hereinafter, the present invention is described in more detail in reference to Examples. However, the scope of the present invention is not limited to these Examples. “%” means “mass %” unless otherwise specified.

### Test Example 1

### Examples 1 to 19, Comparative Examples 1 to 3

(1) Preparation of the film-forming composition

Polyvinyl butyral (manufactured by Sekisui Chemical Co., Ltd.: tradename; S-LEC B BM-1) was used as the component (a) and 99.5% ethanol (manufactured by Wako Pure Chemical Corporation) was used as the component (b) for the film-forming composition. The components shown in Tables 1 to 3 was used as component (c). The content of each component shown in Tables 1 to 3 is an effective amount and the unit is mass %.

### (2) Cosmetic Application Step

A cosmetic shown in Table 4 was applied, in an amount of 200 mg, to an outer side portion (5×5 cm) of a human lower limb with a sponge before an electrostatically spraying step. About 50 mg of the cosmetic was thus applied on the skin.

### (3) Electrostatically Spraying Step

The above film-forming composition was applied by electrostatically spraying to the sites to which the above cosmetics were applied using the electrostatic spraying apparatus 10 having the structure shown in FIG. 1 and the appearance shown in FIG. 2. That is, the above film-forming composition was applied by electrostatic spraying to the skin to which the above cosmetics were applied thereby forming a coating film composed of fibers. The thus formed coating film was pressed by hand from thereon to achieve thorough compatibility between the cosmetic and the coating film.

The conditions under which the film-forming composition was applied by the electrostatic spray were as follows.
Applied site: outer side portion of a human lower limb (5×5 cm) (the site to which the skincare cosmetic was applied)
Electrostatic spraying application time: 30 seconds
Discharge speed of the film-forming composition: 0.1 g/min
Distance between the skin and a nozzle tip from which the film-forming composition is discharged: 80 mm
Application environment: 20° C., 40% RH
Applied voltage: 10 kV

### (4) Foundation Application Step

A foundation was applied to the site to which the cosmetic and the electrostatic spray had been applied. Dermablend Leg & Body Makeup (color: Deep Golden) manufactured by L'Oreal was applied with a sponge. About 20 mg of the cosmetic was thus applied on the skin.

### (5) Electrostatically Spraying Step

The above film-forming composition was applied by the electrostatically spraying to the site to which the cosmetic had been applied using the electrostatic spraying apparatus 10 having the structure shown in FIG. 1 and the appearance shown in FIG. 2. Specifically, the film-forming composition was applied by the electrostatic spray to the skin to which the cosmetic had been applied to form a coating film composed of fibers. The conditions under which the film-forming composition was applied by the electrostatic spray were as follows.
Applied site: outer side portion of the human lower limb (5×5 cm) (the site to which the skincare cosmetic was applied)
Electrostatic spraying application time: 30 seconds
Discharge speed of the film-forming composition: 0.1 g/min
Distance between the skin and a nozzle tip from which the film-forming composition is discharged: 80 mm
Application environment: 20° C., 40% RH
Applied voltage: 10 kV

### (6) Cosmetic Application Step

A milky lotion was applied to the site to which the cosmetic, the electrostatic spraying and the foundation had been applied. A milky lotion shown in Table 5 was applied with a sponge. About 60 mg of the cosmetic was thus applied to the skin.

### (Evaluation of Durability Against Moisture)

The coating films composed of fibers formed by the electrostatic spray on the skin after the skincare cosmetic was applied in Examples 1 to 19 and Comparative Examples 1 to 3 were evaluated for the durability of the coating films against moisture.

The evaluation was implemented as follows. After the film-forming composition was applied onto the cosmetic and the skin by the electrostatic spray to form a coating film composed of fibers thereon, the resultant site was showered with hot water for 2 minutes or more, then the wet site was dried with a towel, and the cosmetic shown in Table 5 was applied to the resultant site and continuously used until the next day, and then the evaluation was conducted.

The evaluation results are shown Tables 1 to 3. The evaluation criteria are as follows.

A: A cosmetic film is neither cracked nor broken.

B: A cosmetic film has a small crack or break smaller than 5 mm.

C: A cosmetic film has a crack or break of 5 mm or larger and smaller than 2 cm.

D: A cosmetic film has a crack or break of 2 cm or larger.

### Test Example 2

### Examples 1 to 19 and Comparative Examples 1 to 3

### (1) Cosmetic Application Step

The cosmetic shown in Table 4 was applied, in an amount of 200 mg, to an inner side portion (5×5 cm) of a human forearm with a sponge before the electrostatically spraying step below. About 50 mg of the cosmetic was thus applied to the skin.
Applied site: inner side portion of a human forearm (5×5 cm)

### (2) Electrostatically Spraying Step

The above film-forming composition was applied by the electrostatically spraying to the site to which the cosmetic had been applied using the electrostatic spraying apparatus 10 having the structure shown in FIG. 1 and the appearance shown in FIG. 2. Specifically, the film-forming composition was applied by the electrostatic spray to the skin to which the cosmetic had been applied to form a coating film composed of fibers.
Applied site: inner side portion of a human forearm (5×5 cm) (the site to which the cosmetic was applied)
Electrostatic spraying application time: 30 seconds
Discharge speed of the film-forming composition: 0.1 g/min
Distance between the skin and a nozzle tip from which the film-forming composition is discharged: 80 mm
Application environment: 30° C., 60% RH
Applied voltage: 10 kV
   (3) Evaluation of Compatibility with Cosmetic

(Compatibility Between the Cosmetic and the Coating Film Composed of Fibers Formed by the Electrostatic Spray)

The coating films composed of fibers formed by the electrostatic spray on the skin after the cosmetics were applied in Examples 1 to 19 and Comparative Examples 1 to 3 were evaluated for the compatibility between the cosmetic shown in Table 4 and the coating films.

The evaluation was conducted by applying the film-forming composition by the electrostatic spraying to the skin immediately after application of the cosmetic thereby forming a coating film composed of fibers, and a state until the cosmetic and the coating film achieved thorough compatibility after complete formation of the coating film was visually observed. The evaluation sites were the inner side of the human forearm. The thorough compatibility of the cosmetics with the coating film herein refers to a state in which the coating film was visually colorless and clear. In the present evaluation, the coating film was never touched by hands or fingers unless otherwise specified. The evaluation results are shown Tables 1 to 3. The evaluation criteria are as follows.

A: Time until the cosmetic and the coating film formed by the electrostatic spraying achieve thorough compatibility is within 1 minute and 30 seconds after complete formation of the coating film.

B: The cosmetic and the coating film do not achieve thorough compatibility within 1 minute and 30 seconds, but the thorough compatibility can be achieved when the coating film is lightly pressed down by hand from thereon.

C: The skincare cosmetic and the coating film do not achieve thorough compatibility within 1 minute and 30 seconds, and the thorough compatibility cannot be achieved even when the coating film is lightly pressed down by hand from thereon.

(4) Evaluation of Softness Obtained after Wetting with Water and Drying

The coating films composed of fibers formed by the electrostatic spray on the skin after the skincare cosmetic was applied in Examples 1 to 13 and Comparative Examples 1 to 3 were evaluated for the influence of moisture on the coating film as softness.

The evaluation was conducted as follows. After the film-forming composition was applied to the skin, immediately after the application of the skincare cosmetic of Table 4, by the electrostatic spraying to form a coating film composed of fibers thereon, the resultant site was showered with water at room temperature for 10 seconds, thereafter, the wet site was dried with a towel, the coating film was waited to be dried, and then, sensory evaluation of softness was conducted. A feel of the film attached to the skin was first evaluated, and then the film was peeled off and pulled to be evaluated. The evaluation criteria are as follows.

A: A pulling feel of the film is not felt when the film is attached to the skin, and when the peeled film is pulled, it stretches and is difficult to break.

B: A pulling feel of the film is slightly felt when the film is attached to the skin, and when the peeled film is pulled, it stretches and is difficult to break.

C: A pulling feel of the film is felt when the film is attached to the skin, and when the peeled film is pulled, it does not stretch but is broken.

D: A pulling feel of the film is strongly felt when the film is attached to the skin, and when the peeled film is pulled, it does not stretch but is broken.

### (5) Evaluation of Followability to Motion of Skin

The coating films composed of fibers formed by the electrostatic spray on the skin in Examples 1 to 19 and Comparative Examples 1 to 3 were evaluated for the followability to motion of the skin.

An evaluation site was set to a region of 5 cm×5 cm on an inner side portion of a human forearm. The electrostatic spray was sprayed for 30 seconds, the resultant was allowed to stand still for 30 seconds, and then a motion for twisting the palm inward and then restoring the palm was repeated 5 times.

The evaluation results are shown Tables 1 to 3. The evaluation criteria are as follows.

A: The coating film follows the skin and no wrinkles are formed.

B: The coating film cannot follow the skin, and small wrinkles are formed in the state where the palm is twisted.

C: The coating film cannot follow the skin and becomes loose in the state where the palm is twisted, and thus wrinkles are formed.

D: The coating film cannot follow the skin, and remains loose even when the palm is restored, and thus wrinkles are formed.

(Elastic Modulus)

The coating films composed of fibers formed by the electrostatic spray on the skin in Examples 1 to 19 and Comparative Examples 1 to 3 were evaluated for the elastic modulus.

TABLE 1

| | | | Example | Example | Example | Example | Example | Example | Example | Example | Example | Example | Example | Example | Example |
|---|---|---|---|---|---|---|---|---|---|---|---|---|---|---|---|
| | Chemical Name | logS | 1 | 2 | 3 | 4 | 5 | 6 | 7 | 8 | 9 | 10 | 11 | 12 | 13 |
| (a) | Polyvinyl Butyral | — | 12.00 | 12.00 | 12.00 | 12.00 | 12.00 | 12.00 | 12.00 | 12.00 | 12.00 | 12.00 | 12.00 | 12.00 | 12.00 |
| | *1 | | | | | | | | | | | | | | |
| (b) | 99.5% Ethanol *2 | — | 83.00 | 83.00 | 83.00 | 83.00 | 83.00 | 83.00 | 83.00 | 83.00 | 83.00 | 83.00 | 83.00 | 84.00 | 80.00 |
| (c) | PPG-7 *3 | −0.9407 | 5.00 | | | | | | | | | | | 4.00 | 8.00 |
| | PPG-9 *4 | −1.472 | | 5.00 | | | | | | | | | | | |
| | PPG-17 *5 | −3.596 | | | 5.00 | | | | | | | | | | |
| | Triethyl Citrate *6 | −0.5133 | | | | 5.00 | | | | | | | | | |
| | Triethyl | −1.402 | | | | | 5.00 | | | | | | | | |
| | Acetylcitrate *7 | | | | | | | | | | | | | | |
| | Tributyl | −3.432 | | | | | | 5.00 | | | | | | | |
| | Acetylcitrate *8 | | | | | | | | | | | | | | |
| | Polyoxyethylene | −2.883 | | | | | | | 5.00 | | | | | | |
| | (20) Sorbitan | | | | | | | | | | | | | | |
| | Monostearate *9 | | | | | | | | | | | | | | |
| | Laureth-3-3 *10 | −3.377 | | | | | | | | 5.00 | | | | | |
| | Ethylhexyl | −4.894 | | | | | | | | | 5.00 | | | | |
| | Methoxycinnamate | | | | | | | | | | | | | | |
| | Oleic Acid | −4.754 | | | | | | | | | | 5.00 | | | |
| | Diethylhexyl | −6.511 | | | | | | | | | | | 5.00 | | |
| | Sebacate *11 | | | | | | | | | | | | | | |
| | Total | | 100.00 | 100.00 | 100.00 | 100.00 | 100.00 | 100.00 | 100.00 | 100.00 | 100.00 | 100.00 | 100.00 | 100.00 | 100.00 |
| | (a) | | 12.00 | 12.00 | 12.00 | 12.00 | 12.00 | 12.00 | 12.00 | 12.00 | 12.00 | 12.00 | 12.00 | 12.00 | 12.00 |
| | (b) | | 82.59 | 82.59 | 82.59 | 82.59 | 82.59 | 82.59 | 82.59 | 82.59 | 82.59 | 82.59 | 82.59 | 83.58 | 79.60 |
| | (c) | | 5.00 | 5.00 | 5.00 | 5.00 | 5.00 | 5.00 | 5.00 | 5.00 | 5.00 | 5.00 | 5.00 | 4.00 | 8.00 |
| | (a)/(b) | | 0.15 | 0.15 | 0.15 | 0.15 | 0.15 | 0.15 | 0.15 | 0.15 | 0.15 | 0.15 | 0.15 | 0.14 | 0.15 |
| | (a)/(c) | | 2.4 | 2.4 | 2.4 | 2.4 | 2.4 | 2.4 | 2.4 | 2.4 | 2.4 | 2.4 | 2.4 | 3.0 | 1.5 |
| | (b)/(c) | | 16.5 | 16.5 | 16.5 | 16.5 | 16.5 | 16.5 | 16.5 | 16.5 | 16.5 | 16.5 | 16.5 | 20.9 | 10.0 |

| | | | | | | | | | | | | | |
|---|---|---|---|---|---|---|---|---|---|---|---|---|---|
| Plasticizing Ability | 1.40E+06 | 1.99E+06 | 6.70E+06 | 1.34E+06 | 2.91E+06 | 2.58E+06 | 6.65E+06 | 1.30E+06 | 2.68E+06 | 7.64E+05 | 8.68E+06 | 1.78E+06 | 2.40E+05 |
| (Elasticity N/m2) | | | | | | | | | | | | | |
| * result of S-S | | | | | | | | | | | | | |
| measurement | | | | | | | | | | | | | |
| Followability to | A | A | A | B | B | A | A | A | B | A | D | A | A |
| Movable Portion | | | | | | | | | | | | | |
| Compatibility with | A | A | B | A | B | A | B | A | B | A | A | A | A |
| Formulation | | | | | | | | | | | | | |
| A: becomes | | | | | | | | | | | | | |
| transparent | | | | | | | | | | | | | |
| to C: not compatible | | | | | | | | | | | | | |
| Softness after Wetting | B | B | A | B | B | B | B | A | A | A | B | B | A |
| Durability (2 days | A | B | A | B | B | B | A | A | B | A | B | A | A |
| after application) | | | | | | | | | | | | | |
| * Result of | | | | | | | | | | | | | |
| Application Test | | | | | | | | | | | | | |
| * A-D | | | | | | | | | | | | | |

TABLE 2

| | Chemical Name | logS | Example 14 | Example 15 | Example 16 | Example 17 | Example 18 | Example 19 |
|---|---|---|---|---|---|---|---|---|
| (a) | Polyvinyl Butyral *1 | — | 15.00 | 9.00 | 12.00 | 12.00 | 12.00 | 16.00 |
| (b) | 99.5% Ethanol *2 | — | 78.75 | 86.00 | 85.00 | 82.00 | 78.00 | 68.00 |
| (c) | PPG-7 *3 | −0.9407 | 6.25 | 5.00 | 3.00 | 6.00 | 10.00 | 6.00 |
| | Neopentyl Glycol Ethylhexanoate | −5.496 | | | | | | 10.00 |
| Total | | | 100.00 | 100.00 | 100.00 | 100.00 | 100.00 | 90.00 |
| (a) | | | 15.00 | 9.00 | 12.00 | 12.00 | 12.00 | 16.00 |
| (b) | | | 78.36 | 85.57 | 84.58 | 81.59 | 77.61 | 67.66 |
| (c) | | | 6.25 | 5.00 | 3.00 | 6.00 | 10.00 | 16.00 |
| (a)/(b) | | | 0.19 | 0.11 | 0.14 | 0.15 | 0.15 | 0.24 |
| (a)/(c) | | | 2.4 | 1.8 | 4.0 | 2.0 | 1.2 | 1.0 |
| (b)/(c) | | | 12.5 | 17.1 | 28.2 | 13.6 | 7.8 | 4.2 |
| | Plasticizing Ability (Elasticity N/m2) | | 4.26E+05 | 5.17E+05 | 2.30E+06 | 9.46E+05 | 1.34E+05 | 1.41E+05 |
| | * result of S-S measurement | | | | | | | |
| | Followability to Movable Portion | | A | A | A | A | A | A |
| | Compatibility with Formulation | | A | A | B | A | A | A |
| | A: becomes transparent | | | | | | | |
| | to C: not compatible | | | | | | | |
| | Softness after Wetting | | A | A | B | B | A | A |
| | Durability (2 days after application) | | A | B | A | B | B | B |
| | * Result of Application Test | | | | | | | |
| | * AD | | | | | | | |

TABLE 3

| | | | Comparative | Comparative | Comparative |
|---|---|---|---|---|---|
| | Chemical Name | logS | Example 1 | Example 2 | Example 3 |
| (a) | Polyvinyl Butyral *1 | | 12.00 | 12.00 | 12.00 |
| (b) | 99.5% Ethanol *2 | | 83.00 | 83.00 | 83.00 |
| others | 1,3-butylene glycol | 0.3559 | 5.00 | | |
| | PEG-8*12 | 1.708 | | 5.00 | |
| | Diisostearyl Malate | −10.86 | | | 5.00 |
| Total | | | 100.00 | 100.00 | 100.00 |
| (a) | | | 12.00 | 12.00 | 12.00 |
| (b) | | | 82.59 | 82.59 | 82.59 |
| (c) | | | — | — | — |
| (a)/(b) | | | 0.15 | 0.15 | 0.15 |
| (a)/(c) | | | — | — | — |
| (b)/(c) | | | — | — | — |
| | Plasticizing Ability (Elasticity N/m2) | | 1.90E+05 | 1.28E+06 | 1.79E+07 |
| | * result of S-S measurement | | | | |
| | Followability to Movable Portion | | A | A | D |
| | Compatibility with Formulation | | A | B | C |
| | A: becomes transparent | | | | |
| | to D: not compatible | | | | |
| | Softness after Wetting | | D | D | C |
| | Durability (2 days after application) | | D | D | C |
| | * Result of Application Test | | | | |
| | * A-D | | | | |

1) S-LEC B BM-1 (manufactured by Sekisui Chemical Co., Ltd.)
2) 99.5% Ethanol (manufactured by Wako Pure Chemical Industries Ltd.)
3) Carpol DL-30 (manufactured by ADEKA Corporation)
4) Polyglycol P-425 (manufactured by The Dow Chemical Company)
5) Carpol DL-80 (manufactured by ADEKA Corporation)
6) Citroflex-2 (manufactured by Morimura Bros., Inc.)
7) Citroflex-A2 (manufactured by Morimura Bros., Inc.)
8) Citroflex-A4 (manufactured by Morimura Bros., Inc.)
9) Rheodol TW-S120V (manufactured by Kao Corporation)
10) GENAPOL LA 030 (manufactured by Clariant Corporation)
11) Fine Neo-EHS (manufactured by Nippon Fine Chemical Co., Ltd.)
12) PEG-400 (manufactured by Sanyo Chemical Industries, Ltd.)

TABLE 4

| Component | mass % |
|---|---|
| Glycerin | 10 |
| Octylacrylamide/hydroxypropyl Acrylate/ | 7 |
| butylaminoethyl Methacrylate Copolymer *13 | |
| Acrylate/alkyl Acrylate (C10-30) | 0.5 |
| Crosspolymer *14 | |
| Ethanol | 4 |
| Aminomethyl Propanol | 1 |
| Mineral Oil | 5 |
| Ceteareth 20 *15 | 0.3 |
| Laureth-3 3 *16 | 0.3 |
| Cetyl Alcohol | 0.3 |
| Stearyl Alcohol | 0.15 |
| Sodium Hydroxide | 0.055 |
| Water | balance |

TABLE 5

| Component | mass % |
|---|---|
| Glycerin | 21 |
| White Petrolatum | 4 |
| Cetearyl Alcohol | 3.5 |
| Behentrimonium Chloride | 1.6 |
| Dimethicone 200 cst | 1.2 |
| Isopropyl Palmitate | 1 |
| Dimethicone 10 cst | 0.5 |
| Benzalkonium Chloride (50 mass %) | 0.2 |
| Water | balance |

13) Amphomer 28-4910 (manufactured by Akzo Nobel N.V.)
14) Pemulen TR-1 (manufactured by Lubrizol Corporation)
15) Eumulgin B2 (manufactured by BASF)
16) GENAPOL LA 030 (manufactured by Clariant Corporation)

Tables 1 to 3 reveal the following: When a composition comprising (a) a polymer having film-forming ability, (b) one or more volatile substances selected from the group consisting of an alcohol and a ketone, and (c) a non-volatile oil having a log S of −7 or more and less than 0 is applied directly to the skin by the electrostatic spraying, a coating film composed of fibers is formed on the skin, and the coating film has a favorable elastic modulus, and is excellent in the skin compatibility, the adhesion, the followability to motion of the skin, and the durability against moisture.

### REFERENCE SIGNS LIST

10 Electrostatic spraying apparatus
11 Low-voltage power supply
12 High-voltage power supply
13 Auxiliary electric circuit
14 Micro gear pump
15 Container
16 Nozzle
17 Pipeline
18 Flexible pipeline
19 Electric current limiting resistor
20 Housing

1: A film-forming composition, for forming a coating film composed of fibers directly on the skin by electrostatic spraying, wherein the composition comprises the following components (a), (b) and (c), and the coating film formed from the film-forming composition has an elastic modulus of 1×10⁴ N/m² or more and 9×10⁶ N/m² or less:
(a) a polymer having film-forming ability;
(b) one or more volatile substances selected from the group consisting of an alcohol and a ketone; and
(c) 2 mass % or more and 30 mass % or less of a non-volatile oil having a log S of −7 or more and less than 0.

2: The film-forming composition according to claim 1, wherein the component (c) has a log S of −6 or more and less than 0.

3: The film-forming composition according to claim 1, wherein a content mass ratio of the component (a) to the component (c), ((a)/(c)), is 0.15 or more and 300 or less.

4: The film-forming composition according to claim 1, wherein a content mass ratio of the component (b) to the component (c), ((b)/(c)), is 0.80 or more and 200 or less.

5: The film-forming composition according to claim 1, wherein a content mass ratio of the component (a) to the component (b), ((a)/(b)), is 0.03 or more and 0.6 or less.

6: The film-forming composition according to claim 1, wherein a content of the component (a) is 4 mass % or more and 35 mass % or less, and a content of the component (b) is 45 mass % or more and 95 mass % or less.

7: The film-forming composition according to claim 1, wherein a content of the component (a) is 4 mass % or more and 30 mass % or less, and a content of the component (b) is 50 mass % or more and 93 mass % or less.

8: The film-forming composition according to claim 1, wherein a content of the component (c) is 2 mass % or more and 25 mass % or less.

9: The film-forming composition according to claim 1, wherein the (a) polymer having film-forming ability is at least one selected from the group consisting of a partially saponified polyvinyl alcohol, a low saponified polyvinyl alcohol, a completely saponified polyvinyl alcohol, a polyvinyl butyral resin, an alkyl acetalized polyvinyl alcohol, a polyurethane resin, a polymethacrylate resin, an oxazoline-modified silicone, a polyvinyl acetal diethylamino acetate, and a polylactic acid.

10: The film-forming composition according to claim 1, wherein the component (c) is one or more selected from the group consisting of a poly-C3-C4 alkylene glycol, a polyoxyalkylene alkyl ether, an ester oil, a silicone oil, a nonionic (non-ionic) surfactant, and a higher fatty acid.

11: The film-forming composition according to claim 1, which is used in combination with a cosmetic applied to the skin by a device other than an electrostatic spray.

12: A film-forming composition, for forming a coating film composed of fibers directly on the skin by electrostatic spraying, wherein the composition comprises the following components (a), (b) and (c), and the coating film formed from the film-forming composition has an elastic modulus of 1×10⁴ N/m² or more and 9×10⁶ N/m² or less:
(a) 4 mass % or more and 35 mass % or less of a polymer having film-forming ability;
(b) 45 mass % or more and 95 mass % or less of one or more volatile substances selected from the group consisting of an alcohol and a ketone; and
(c) 2 mass % or more and 30 mass % or less of a non-volatile oil having a log S of −7 or more and less than 0.

13: A method for producing a coating film, for forming a coating film on a surface of a coating film formation subject, the method comprising electrostatically spraying for forming a coating film composed of deposits comprising fibers by directly electrostatically spraying a composition on the surface of the coating film formation subject,
wherein the composition comprises the following components (a), (b) and (c), and the coating film formed from the composition has an elastic modulus of 1×10⁴ N/m² or more and 9×10⁶ N/m² or less:
(a) 4 mass % or more and 35 mass % or less of a polymer having film-forming ability;
(b) 45 mass % or more and 95 mass % or less of one or more volatile substances selected from the group consisting of an alcohol and a ketone; and
(c) 2 mass % or more and 30 mass % or less of a non-volatile oil having a log S of −7 or more and less than 0.

14: A cosmetic comprising a film-forming composition for forming a coating film composed of fibers directly on the skin by electrostatic spraying, wherein the composition comprises the following components (a), (b) and (c), and the coating film formed from the film-forming composition has an elastic modulus of 1×10⁴ N/m² or more and 9×10⁶ N/m² or less:
(a) 4 mass % or more and 35 mass % or less of a polymer having film-forming ability;
(b) 45 mass % or more and 95 mass % or less of one or more volatile substances selected from the group consisting of an alcohol and a ketone; and
(c) 2 mass % or more and 30 mass % or less of a non-volatile oil having a log S of −7 or more and less than 0.
